(19) United States
(12) Patent Application Publication
(10) Pub. No.: US 2017/0202770 A1
(43) Pub. Date: Jul. 20, 2017

(54) COMPOSITION COMPRISING POLYGLYCEROL ESTERS AND HYDROXYALKYL-MODIFIED GUAR

(71) Applicant: Evonik Degussa GmbH, Essen (DE)

(72) Inventors: Achim Friedrich, Hattingen (DE); Juergen Meyer, Essen (DE); Frank Unger, Oberhausen (DE); Verena Dahl, Kurten (DE); Jan Marian von Hof, Essen (DE); Oliver Springer, Wesel (DE)

(21) Appl. No.: 15/380,137

(22) Filed: Dec. 15, 2016

(30) Foreign Application Priority Data
Jan. 15, 2016 (EP) 16151443.5

(51) Int. Cl. A61K 8/85; A61K 8/26; A61Q 15/00; A61Q 1/02; A61Q 17/04; A61Q 19/00; A61Q 19/08; A61K 8/73; A61K 8/06

(52) U.S. Cl. CPC A61K 8/85; A61K 8/737; A61K 8/26; A61Q 15/00; A61K 8/062; A61Q 17/04; A61Q 19/007; A61Q 19/08; A61Q 1/02; A61K 2800/30

(57) ABSTRACT

The invention relates to compositions comprising polyglycerol esters and hydroxyalkyl-modified guar.

### FIELD OF THE INVENTION

The invention relates to compositions comprising polyglycerol esters and long-chain hydroxyalkyl-modified guar.

### PRIOR ART

WO2013092186 discloses an antiperspirant composition in the form of an oil-in-water emulsion, which is not a microemulsion, comprising

a) at least one antiperspirant aluminum salt in a total amount of 2 -40% by weight, wherein the percentages by weight refer to the total weight of the water of crystallization-free and ligand-free active substance (USP) in the composition,

and in addition to this

b) at least one surface-active compound, in a total amount of 0.1 -2% by weight, having an HLB value in the range of 9 to 15, selected from the partial esters of a polyglycerol, comprising 3, 4 or 5 glycerol units, with a linear or branched, saturated or unsaturated carboxylic acid having 8 to 22 carbon atoms and with an organic food acid,

and in addition to this

c) at least one particular N-acyl-L-glutamic acid sodium salt in a total amount of 0.1 -2.0% by weight.

The object of the invention was to provide compositions which enable a low-viscosity and stable formulation of emulsion-burdening ingredients such as antiperspirant/deodorant active ingredients.

### DESCRIPTION OF THE INVENTION

Surprisingly, it has been found that the compositions described below are able to solve the problem addressed by the invention.

The invention therefore relates to compositions comprising certain polyglycerol esters and hydroxyalkyl-modified guar.

One advantage of the present invention is that the polyglycerol ester present in the compositions according to the invention is based completely on renewable raw materials.

Another advantage of the present invention is that the composition according to the invention is suitable for the formulation of O/W emulsions (creams, lotions) with excellent storage stability.

A further advantage of the present invention is that the composition according to the invention is suitable for the formulation of PEG-free emulsions, in particular mobile PEG-free emulsions.

A further advantage of the present invention is that the composition according to the invention is suitable for the formulation of PEG-free antiperspirant/deodorant emulsions, in particular roll-on emulsions.

A further advantage of the present invention is that the composition according to the invention is suitable for the formulation of emulsions having a yield point.

Emulsions and formulations comprising such emulsifier based on the composition according to the invention moreover have a good skinfeel.

Advantageously, emulsions and formulations comprising the composition according to the invention require no paraben-containing preservatives.

A further advantage of the present invention is that the composition according to the invention is suitable for the formulation of emulsions without polyacrylate-based thickeners.

A further advantage of the present invention is that the composition according to the invention can be handled easily on account of its consistency.

A further advantage of the present invention is that the composition according to the invention produces a light skinfeel in formulations.

A further advantage of the present invention is that the use of the composition according to the invention imparts moisturizing properties to the formulations.

A composition is therefore claimed comprising

A) polyglycerol ester which, after its complete hydrolysis, releases

a) at least one carboxylic acid having 6 to 14, preferably 8 to 12, carbon atoms,

b) at least one carboxylic acid having 16 to 20, preferably 18 to 20, carbon atoms,

c) at least one carboxylic acid having 22 to 28, preferably 22 to 24, carbon atoms,

and

which, after its complete hydrolysis, releases polyglycerol having an average degree of polymerization of from 3.0 to 5.0, preferably from 3.3 to 4.7, particularly preferably from 3.6 to 4.5,

and

B) at least one hydroxyalkyl-modified guar,

characterized in that the hydroxyalkyl-modified guar has been modified with hydroxyalkyl groups having 12 to 26, preferably 16 to 24, particularly preferably 18 to 22 carbon atoms.

In the context of the present invention, the term “polyglycerol” is to be understood as meaning a polyglycerol which comprises glycerol. Consequently, for the purposes of calculating amounts, masses and the like, the glycerol fraction should also be taken into consideration. In the context of the present invention, polyglycerols are therefore mixtures of glycerol and at least one glycerol oligomer. Glycerol oligomers are to be understood in each case as meaning all corresponding structures, i.e., for example, linear and cyclic compounds.

The same applies to the term “polyglycerol ester” in connection with the present invention.

The stated number-average of the acid residues, in the case of more than one of carboxylic acid a), b) or c), refers in each case to the accumulated sum of all carboxylic acids a), b) or c).

The average degree of polymerization of the polyglycerol N is calculated via its hydroxyl number (OHN, in mg KOH/g) according to the following formula:

$$N = \frac{( 112200 - 18 \cdot OHN )}{( 74 \cdot OHN - 56100 )}$$

Suitable methods for determining the hydroxyl number are particularly those according to DGF C-V 17 a (53), Ph. Eur. 2.5.3 Method A and DIN 53240.

Unless otherwise stated, all percentages (%) given are percentages by weight.

Preferred compositions according to the invention are those in which the polyglycerol released after complete hydrolysis of the polyglycerol ester has a mass ratio of glycerol to diglycerol of greater than 1, preferably greater than 1.2, particularly preferably greater than 1.4.

Particularly preferred compositions according to the invention are those in which the polyglycerol released after complete hydrolysis of the polyglycerol ester according to the invention comprises

5% by weight to 30% by weight, preferably 7% by weight to 25% by weight, particularly preferably 10% by weight to 22% by weight, monoglycerol,

1% by weight to 25% by weight, preferably 3% by weight to 18% by weight, particularly preferably 5% by weight to 15% by weight, diglycerols,

1% by weight to 25% by weight, preferably 1% by weight to 20% by weight, particularly preferably 3% by weight to 17% by weight, triglycerols and

1% by weight to 20% by weight, preferably 2% by weight to 15% by weight, particularly preferably 4% by weight to 10% by weight, tetraglycerols,

where the percentages by weight refer to the total polyglycerol.

In this context, the polyglycerol released preferably has

≧70% by weight, preferably ≧75% by weight, particularly preferably ≧80% by weight, polyglycerols having a degree of polymerization of ≧2,

≧60% by weight, preferably ≧65% by weight, particularly preferably ≧70% by weight, polyglycerols having a degree of polymerization of ≧3,

≧50% by weight, preferably ≧55% by weight, particularly preferably ≧60% by weight, polyglycerols having a degree of polymerization of ≧4 and

≧40% by weight, preferably ≧45% by weight, particularly preferably ≧50% by weight, polyglycerols having a degree of polymerization of ≧5,

where the percentages by weight refer to the total polyglycerol.

The mass fraction of glycerol, diglycerol, triglycerol, tetraglycerol and of the fatty acids can be determined for the purposes of the present invention by two GC methods; these methods include the alkaline hydrolysis of the polyglycerol ester according to the invention, separation of the polyglycerol from the acids released and analysis of the fatty acids, and also of the glycerol oligomers (linear and cyclic).

For this, 0.5 g of the polyglycerol ester according to the invention is boiled in 25 ml of an ethanolic 0.5 M KOH solution under reflux for 4 hours. Then, 10 ml of water are added and the pH is adjusted to pH 2-3 with sulphuric acid. The resulting carboxylic acids are separated off by means of extraction three times with one volume (20 ml) of petroleum ether each time.

Fatty acid analysis:

The combined extracts are concentrated to about 1 ml by evaporation.

Suitable determination methods for ascertaining the fatty acid distribution are in particular those according to DGF C VI 11a, DGF C-VI 10a and GAT—ring test 7/99.

A 0.5 ml aliquot of the petroleum ether extract obtained as described above is treated in a vessel with 1 ml of a mixture of acetyl chloride:methanol (1:4) at boiling point for 30 min with exclusion of atmospheric moisture. The resulting fatty acid methyl esters are extracted twice with 5 ml of isooctane each time and analysed by GC. This is carried out in a gas chromatograph equipped with a split/splitless injector, a capillary column and a flame ionization detector, under the following conditions:
Injector: 290° C., split 30 ml
Injection volume: 1 μl
Column: 30 m *0.32 mm HP1 0.25 μm
Carrier gas: Helium, constant flow, 2 ml/min
Temperature programme: 80° C.-300° C. at 8° C./min, then
   conditioning for 10 minutes at 300° C.
Detector: FID at 320° C.
   Hydrogen 35 ml/min
   Air 240 ml/min
   Make-up gas 12 ml/min

The carboxylic acids are separated as their methyl esters according to their carbon chain length and their mass fraction is determined according to an internal standard method. For this, the GC system is calibrated by analysing fatty acid methyl ester mixtures of the fatty acids to be investigated with known composition.

Using this method, the total mass and the mass fractions of carboxylic acid(s) are obtained, which permit a determination of the molar amount(s) by using the respective molecular weights. The total mass of carboxylic acid(s) can moreover be used to determine, by means of subtraction, the mass of polyglycerol present, for example, in 0.5 g of polyglycerol ester.

Using the molecular weight of the polyglycerol, the molar amount of the polyglycerol can be determined therefrom.

| | | |
|---|---|---|
| Mₚ = 74 · N + 18 | with | Mₚ = molecular weight of the polyglycerol |
| | | [g/mol] |
| | | N = degree of polymerization of the |
| | | polyglycerol (as regards the determination |
| | | of the degree of polymerization, |
| | | see below). |
| ${n}_{p} = \frac{{m}_{p}}{{M}_{p}}$ | with | nₚ = molar amount of the polyglycerol [mol] in 1 g of polyglycerol ester mₚ = mass of polyglycerol in 1 g of |
| | | polyglycerol ester [g] |
| | | Mₚ = molecular weight of the polyglycerol |
| | | [g/mol] |

Together, the molar ratios of polyglycerol to carboxylic acids can be determined from these values.

Analysis of glycerol, diglycerols, triglycerols and tetraglycerols:

The residue extracted with petroleum ether is adjusted with barium hydroxide to pH 7 to 8. The precipitated barium sulphate is separated off by centrifugation.

The supernatant is drawn off and the residue is extracted three times with 20 ml of ethanol.

The combined supernatants are concentrated for 30 min at 80° C. and 50 mbar and dried.

For the analysis of glycerol, diglycerols, triglycerols and tetraglycerols by means of GC, the residue is dissolved in 2 ml of pyridine:chloroform (4:1). 0.5 ml of this solution is admixed with 1 ml of MSTFA [N-methyl-N-(trimethylsilyl)trifluoroacetamide]. The alcohols are quantitatively converted to their trimethylsilyl ethers by reaction at 80° C. (30 minutes) and then analysed by means of GC/FID.

This is carried out in a gas chromatograph equipped with a split/splitless injector, a capillary column and a flame ionization detector, under the following conditions:
Injector: 290° C., split 30 ml
Injection volume: 1 μl
Column: 30 m *0.32 mm HP1 0.25 μm
Carrier gas: Helium, constant flow, 2 ml/min
Temperature programme: 80° C.-300° C. at 4° C./min, then
   conditioning for 10 minutes at 300° C.
Detector: FID at 310° C.
   Hydrogen 35 ml/min
   Air 240 ml/min
   Make-up gas 12 ml/min

Glycerol, diglycerols, triglycerols and tetraglycerols are separated and their mass fraction is determined by an internal standard method. For this, the GC system is calibrated by analysing mixtures of the glycerols to be investigated and of the internal standard with known composition.

The mass fractions can be used to determine the mass ratio of glycerol to diglycerol and, by subtraction from 100%, also the content of polyglycerols with a degree of polymerization of 2 and greater (100% minus mass fraction of the glycerol), the content of polyglycerols with a degree of polymerization of 3 and greater (100% minus mass fractions of the glycerol and of the diglycerols), the content of polyglycerols with a degree of polymerization of 4 and greater (100% minus mass fractions of the glycerol, the diglycerols and the triglycerols) and the content of polyglycerols with a degree of polymerization of 5 and greater (100% minus mass fractions of the glycerol, the diglycerols, the triglycerols and the tetraglycerols).

Should glycerol, but no detectable amount of diglycerol, be present in a polyglycerol under consideration, then this corresponds to a mass ratio of glycerol to diglycerol of greater than 1.4.

A preferred composition according to the invention is characterized in that the polyglycerol ester, after its complete hydrolysis, releases an average (number average) per mole of polyglycerol ester of

from 0.01 to 0.07 mol, preferably from 0.01 to 0.50 mol, particularly preferably from 0.01 to 0.30 mol, of at least one carboxylic acid a)

from 0.10 to 1.70 mol, preferably from 0.30 to 1.50 mol, particularly preferably from 0.40 to 1.40 mol, of at least one carboxylic acid b)

from 0.01 to 0.80 mol, preferably from 0.01 to 0.60 mol, particularly preferably from 0.05 to 0.40 mol, of at least one carboxylic acid c).

In particular, said composition is characterized in that the molar ratio of carboxylic acid a) to carboxylic acid b) to carboxylic acid c) obtained after complete hydrolysis of the polyglycerol ester is

0.6 to 1.4:16.5 to 20.5:3.0 to 4.8, preferably

0.8 to 1.2:17.5 to 19.5:3.5 to 4.3, particularly preferably

0.9 to 1.1:18.0 to 19.0:3.7 to 4.1.

A method for determining the molar ratios that can be used is the method described above.

It is preferred according to the invention that the carboxylic acids a), b) and c) are selected from fatty acids, these being in particular selected from linear, saturated, unsubstituted carboxylic acids.

In particular, preference is given to compositions according to the invention which are characterized in that carboxylic acid a) is selected from caprylic acid and capric acid, carboxylic acid b) is selected from stearic acid and palmitic acid and carboxylic acid c) is behenic acid.

Hydroxyalkyl-modified guar has been described many times and is commercially available, for example, as Esaflor HM 22. Guar is a galactomannan in which long-chain hydroxyalkyl modifications may be inserted simply by reacting with epoxyalkanes.

Preferred compositions in accordance with invention comprise hydroxyalkyl-modified guar which has been modified with alkyl groups having 16 to 24, preferably 18 to 22, carbon atoms.

It is preferred in accordance with the invention that the hydroxyalkyl-modified guar has additionally been hydroxypropyl-modified. A guar particularly preferably present in accordance with the invention is the substance named by INCI as C18-C22 Hydroxyalkyl Hydroxypropyl Guar.

Hydroxyalkyl-modified guar and the same which has been additionally hydroxypropyl-modified and also methods for preparation of these compounds are described, for example, in U.S. Pat. No.4,960,876.

The compositions according to the invention are outstandingly suitable for formulating deodorant or antiperspirant-deodorant compositions; therefore, they preferably additionally comprise

C) at least one active ingredient selected from deodorant active ingredient and antiperspirant active ingredient, in particular at least one deodorant active ingredient and at least one antiperspirant active ingredient.

It is preferred in accordance with the invention that the antiperspirant active ingredient is selected from the group comprising, preferably consisting of, aluminium salts and zirconium salts.

In particular, preference is given to compositions according to the invention which are characterized in that the aluminium salt is selected from the group comprising, preferably consisting of:

Aluminum Acetate, Aluminum Behenate, Aluminum Benzoate, Aluminum Bromohydrate, Aluminum Butoxide, Aluminum Calcium Sodium Silicate, Aluminum Caprylate, Aluminum Capryloyl Hydrolyzed Collagen, Aluminum Chloride, Aluminum Chlorohydrate, Aluminum Chlorohydrex, Aluminum Chlorohydrex PEG, Aluminum Chlorohydrex PG, Aluminum Citrate, Aluminum Diacetate, Aluminum Dibenzoate/Stearate Hydroxide, Aluminum Dicetyl Phosphate, Aluminum Dichlorohydrate, Aluminum Dichlorohydrex PEG, Aluminum Dichlorohydrex PG, Aluminum Dilinoleate, Aluminum Dimyristate, Aluminum Distearate, Aluminum Glycinate, Aluminum Hydrogenated Tallow Glutamate, Aluminum Hydroxy Bis-Methylene Bis-Di-t-Butylphenyl Phosphate, Aluminum Iron Calcium Magnesium Germanium Silicates, Aluminum Iron Calcium Magnesium Zirconium Silicates, Aluminum Iron Silicates, Aluminum Isopropoxide, Aluminum Isostearate, Aluminum Isostearates/Laurates/Palmitates, Aluminum Isostearates/Laurates/Stearates, Aluminum Isostearates/Myristates, Aluminum Isostearates/Palmitates, Aluminum Isostearates/Stearates, Aluminum Isostearyl Glyceryl Phosphate, Aluminum Laccate, Aluminum Lactate, Aluminum Lanolate, Aluminum/Magnesium Hydroxide Stearate, Aluminum Magnesium Oxide, Aluminum Methionate, Aluminum Myristate, Aluminum Myristates/Palmitates, Aluminum PCA, Aluminum Phenolsulfonate, Aluminum Sesquichlorohydrate, Aluminum Sesquichlorohydrex PEG, Aluminum Sesquichlorohydrex PG, Aluminum Starch Octenylsuccinate, Aluminum Stearate, Aluminum Stearates, Aluminum Stearoyl Glutamate, Aluminum Sucrose Octasulfate, Aluminum Sulfate, Aluminum Triformate, Aluminum Triphosphate, Aluminum Tristearate, Aluminum Undecylenoyl Collagen Amino Acids, Aluminum Zinc Oxide, Aluminum Zirconium Trichlorohydrate, Aluminum Zirconium Trichlorohydrex GLY, Aluminum Zirconium Tetrachlorohydrate, Aluminium Zirconium Tetrachlorohydrex GLY,

Aluminum Zirconium Tetrachlorohydrex PEG, Aluminum Zirconium Tetrachlorohydrex PG, Aluminum Zirconium Pentachlorohydrate, Aluminum Zirconium Pentachlorohydrex GLY, Aluminum Zirconium Octachlorohydrate, Aluminum Zirconium Octachlorohydrex GLY, Ammonium Alum, Ammonium Silver Zinc Aluminum Silicate, Calcium Aluminum Borosilicate, Cobalt Aluminum Oxide, Magnesium/Aluminum/Hydroxide/Carbonate, Magnesium Aluminum Silicate, Magnesium/Aluminum/Zinc/Hydroxide/Carbonate, Potassium Alum, Potassium Aluminum Polyacrylate, Silver Magnesium Aluminum Phosphate, Sodium Alum, Sodium Aluminate, Sodium Aluminum Chlorohydroxy Lactate, Sodium/Aluminum Hydroxide/Oxalate/Sulfate, Sodium/Aluminum/Iron Hydroxide/Oxalate/Sulfate, Sodium/Aluminum/Iron/Sulfate/Citrate/Hydroxide, Sodium/Aluminum/Iron/Sulfate/Oxalate/Hydroxide, Sodium/Aluminum/Iron/Sulfate/Tartarate/Hydroxide, Sodium Aluminum Lactate, Sodium Phosphorus/Zinc/Calcium/Silicon/Aluminum/Silver Oxides, Sodium Potassium Aluminum Silicate, Sodium Silicoaluminate, Sodium Silver Aluminum Silicate, Tromethamine Magnesium Aluminum Silicate and Alum, in particular aluminium chlorohydrate.

In the context of the present invention, the term “aluminium chlorohydrate” is understood to mean the salts of the general empirical formula AlₙCl₍₃ₙ₋ₘ₎(OH)ₘ in particular Al₂Cl(OH)₅.

Suitable zirconium salts are selected from the compounds containing zirconium described in US20070071701, U.S. Pat. No.3,792,068 and U.S. Pat. No.2,854,382.

Suitable deodorant active ingredients are selected from the group comprising, preferably consisting of, deodorants and microbe-inhibiting agents mentioned in EP2421614, and zinc salts.

The zinc salt preferred in accordance with the invention is selected from the group of the zinc salts of acetic acid, lactic acid, oxalic acid, succinic acid, fumaric acid, maleic acid, ricinoleic acid and/or citric acid.

The compositions according to the invention particularly stabilize emulsions such that a composition preferred according to the invention is characterized in that it is an emulsion, particularly an oil-in-water emulsion.

Emulsions preferred according to the invention comprise from 0.1% by weight to 60.0% by weight, preferably from 4.0% by weight to 30.0% by weight, particularly preferably from 5.0% by weight to 20.0% by weight, based on the total emulsion, of at least one oil, particularly a cosmetic oil.

Suitable cosmetic oils are listed, for example, in DE10361202.

For good emulsion stability, it is preferred that the emulsions according to the invention comprise from 0.1% by weight to 15.0% by weight, preferably from 0.5% by weight to 10.0% by weight, particularly preferably from 1.0% by weight to 7.0% by weight, based on the total emulsion, of at least one emulsifier.

Preferred emulsifiers present are particularly oil-in-water emulsifiers, in particular emulsifiers listed in EP2421614.

The compositions according to the invention even stabilize normally difficult formulations having a low viscosity and therefore a preferred composition according to the invention is characterized in that it has a viscosity in a range from 500 to 20 000, preferably from 1500 to 10 000 mPas, where the viscosity is measured at 25° C. using a Brookfield RVT, spindle 4, 5 rpm.

Particular preference is given to compositions which are essentially polyglycol ether-free and essentially free of alkoxylated compounds. The terms “essentially free of alkoxylated compounds” and “essentially polyglycol ether-free”, in connection with the present invention, are understood to mean that the compositions, if appropriate with component B present according to the invention as an exception, have no significant amounts of alkoxylated compounds or compounds comprising polyglycol ethers which exert a surface-active effect. This is particularly understood to mean that these compounds are present in amounts of less than 1% by weight, preferably less than 0.1% by weight, particularly preferably less than 0.01% by weight, based on the total composition, in particular no detectable amounts.

The examples presented below illustrate the present invention by way of example, without any intention of restricting the invention, the scope of application of which is apparent from the entirety of the description and the claims, to the embodiments specified in the examples.

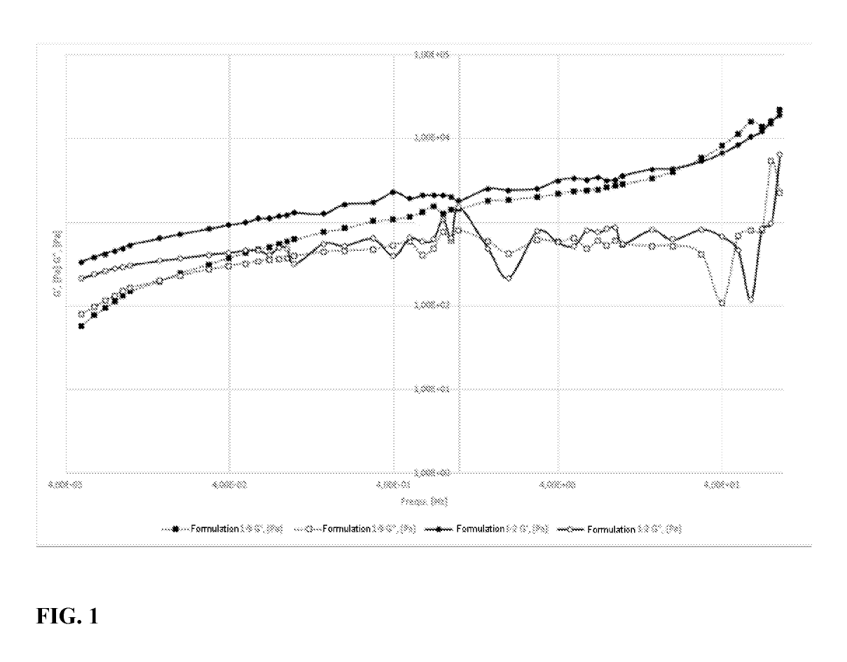

FIG. 1 shows storage modulus and loss modulus for the emulsions prepared according to inventive formulation 1-2 or non-inventive formulation 1-9.

### EXAMPLES

### Presynthesis Example 1

### Glyceryl monobehenate

A mixture of glycerol (92 g, 1.0 mol), behenic acid (234.6 g, 0.69 mol) and Ca(OH)₂ (0.06 g) was heated up to 240° C. over the course of 3 h with the introduction of nitrogen and the mixture was then stirred at this temperature and the water which formed was continuously removed until an acid number of ≦1.5 was reached. After cooling to 90° C., a 5% solution of H₃PO₄ in glycerol (2 g) was then added and the reaction mixture heated again to 240° C. From a temperature of ≧100° C., the pressure was decreased to 10 mbar with simultaneous nitrogen introduction and the mixture was distilled until no more distillate was obtained. After addition of a filter aid, the mixture is filtered through a filter.

### Presynthesis Example 2

### Glyceryl monostearate

A mixture of glycerol (198.3 g, 2.15 mol), stearic acid and palmitic acid in a 1:1 ratio (401.8 g, 1.49 mol) and Ca(OH)₂ (0.13 g) was heated up to 240° C. over the course of 3 h with the introduction of nitrogen and the mixture was then stirred at this temperature and the water which formed was continuously removed until an acid number of ≦1.5 was reached. After cooling to 90° C., a 5% solution of H₃PO₄ in glycerol (4.3 g) was then added and the reaction mixture was heated again to 240° C. From a temperature of ≧100° C., the pressure was decreased to 10 mbar with simultaneous nitrogen introduction and the mixture was distilled until no more distillate was obtained. After addition of a filter aid, the mixture is filtered through a filter.

### Presynthesis Example 3

### Polyglyceryl-4 stearate

A mixture of glycerol (2102 g, 22.8 mol) and 45% aqueous potassium hydroxide solution (24.2 g) was heated to 240° C. at 400 mbar over the course of 1 hour and the water which formed was continuously distilled off. As soon as the reaction mixture had reached a refractive index of ≦1.4830, the pressure was slowly reduced to 50 mbar and further water and excess glycerol were distilled off at 240° C. until the remaining mixture had a hydroxyl number of 990 mg KOH/g.

A mixture of the polyglycerol thus obtained (252.2 g, 0.8 mol) and stearic acid and palmitic acid in a ratio of 1:1 (97.8 g, 0.36 mol) was heated up to 240° C. over the course of 3 h with the introduction of nitrogen and the mixture was then stirred at this temperature and the water which formed was continuously removed until an acid number of ≦1.0 was reached and the mixture was clear and homogeneous at 240° C.

### Presynthesis Example 4

### Polyglyceryl-3 caprylate/caprate

A mixture of commercially available polyglycerol-3 (Solvay; 240 g, 1 mol) and caprylic acid and capric acid in a ratio of 60:40 (78.8 g, 0.5 mol) was heated up to 240° C. over the course of 3 h with the introduction of nitrogen and the mixture was then stirred at this temperature and the water which formed was continuously removed until an acid number of ≦0.5 was reached.

### Synthesis Example 1

### Non-Inventive

A mixture of esters obtained as described in presynthesis example 1 (22.5 g), in presynthesis example 2 (34.5 g), in presynthesis example 3 (88.5 g) and in presynthesis example 4 (4.5 g) was heated to 80° C. and the mixture was then stirred at this temperature for 3 h.

The polyglycerol ester thus obtained, after its complete hydrolysis, has a degree of polymerization of the polyglycerol of ≦3.

### Presynthesis Example 5

### Polyglyceryl-6 stearate

A mixture of glycerol (2102 g, 22.8 mol) and 45% aqueous potassium hydroxide solution (24.2 g) was heated to 240° C. at 400 mbar over the course of 1 hour and the water which formed was continuously distilled off. As soon as the reaction mixture had reached a refractive index of ≧1.4830, the pressure was slowly reduced to 50 mbar and further water and excess glycerol were distilled off at 240° C. until the remaining mixture had a hydroxyl number of 960 mg KOH/g.

A mixture of the polyglycerol thus obtained (1008.7 g, 2.2 mol) and stearic acid and palmitic acid in a ratio of 1:1 (391.3 g, 1.5 mol) was heated up to 240° C. over the course of 3 h with the introduction of nitrogen and the mixture was then stirred at this temperature and the water which formed was continuously removed until an acid number of ≦1.0 was reached and the mixture was clear and homogeneous at 240° C.

### Synthesis Example 2

### Inventive

A mixture of esters obtained as described in presynthesis example 1 (18.75 g), in presynthesis example 2 (32.25 g), in presynthesis example 5 (94.5 g) and in presynthesis example 4 (4.5 g) was heated to 80° C. and the mixture was then stirred at this temperature for 3 h.

The polyglycerol ester thus obtained, after its complete hydrolysis, has a degree of polymerization of the polyglycerol of ca. 3.9.

### Presynthesis Example 6

### Polyglyceryl-10 stearate

A mixture of glycerol (2102 g, 22.8 mol) and 45% aqueous potassium hydroxide solution (24.2 g) was heated to 240° C. at 400 mbar over the course of 1 hour and the water which formed was continuously distilled off. As soon as the reaction mixture had reached a refractive index of ≦1.4830, the pressure was slowly reduced to 50 mbar and further water and excess glycerol were distilled off at 240° C. until the remaining mixture had a hydroxyl number of 875 mg KOH/g.

A mixture of the polyglycerol thus obtained (252.2 g, 0.33 mol) and stearic acid and palmitic acid in a ratio of 1:1 (97.8 g, 0.36 mol) was heated up to 240° C. over the course of 3 h with the introduction of nitrogen and the mixture was then stirred at this temperature and the water which formed was continuously removed until an acid number of ≦1.0 was reached and the mixture was clear and homogeneous at 240° C.

### Presynthesis Example 7

### Polyglyceryl-10 caprylate/caprate

A polyglycerol obtained as described in example 5a (240 g, 0.32 mol) and caprylic acid and capric acid in a ratio of 60:40 (78.8 g, 0.5 mol) was heated up to 240° C. over the course of 3 h with the introduction of nitrogen and the mixture was then stirred at this temperature and the water which formed was continuously removed until an acid number of ≦1.0 was reached.

### Synthesis Example 3

### Inventive

A mixture of esters obtained as described in presynthesis example 1 (18.8 g), in presynthesis example 2 (32.7 g), in presynthesis example 6 (93.8 g) and in presynthesis example 7 (4.7 g) was heated to 80° C. and the mixture was then stirred at this temperature for 3 h.

The polyglycerol ester thus obtained, after its complete hydrolysis, has a degree of polymerization of the polyglycerol of ca. 4.5.

### Presynthesis Example 8

A mixture of glycerol (2102 g, 22.8 mol) and 45% aqueous potassium hydroxide solution (24.2 g) was heated to 240° C. at 400 mbar over the course of 1 hour and the water which formed was continuously distilled off. As soon as the reaction mixture had reached a refractive index of ≧1.4830, the pressure was slowly reduced to 50 mbar and further water and excess glycerol were distilled off at 240° C. until the remaining mixture had a hydroxyl number of 924 mg KOH/g.

A mixture of the polyglycerol thus obtained (231.9 g, 0.296 mol) and stearic acid and palmitic acid in a ratio of 1:1 (85.42 g, 60 mol) and caprylic acid and capric acid in a ratio of 60:40 (3.82 g, 0.025 mol) was heated up to 240° C. over the course of 3 h with the introduction of nitrogen and the mixture was then stirred at this temperature and the water which formed was continuously removed until an acid number of ≦1.0 was reached and the mixture was clear and homogeneous at 240° C.

### Synthesis Example 4

### Inventive

A mixture of esters obtained as described in presynthesis example 1 (18.75 g), in presynthesis example 2 (33.75 g) and in presynthesis example 8 (97.5 g) was heated to 80° C. and the mixture was then stirred at this temperature for 3 h.

The polyglycerol ester thus obtained, after its complete hydrolysis, has a degree of polymerization of the polyglycerol of ca. 3.6.

### Presynthesis Example 9

A mixture of glycerol (2102 g, 22.8 mol) and 45% aqueous potassium hydroxide solution (24.2 g) was heated to 240° C. at 400 mbar over the course of 1 hour and the water which formed was continuously distilled off. As soon as the reaction mixture had reached a refractive index of ≧1.4830, the pressure was slowly reduced to 50 mbar and further water and excess glycerol were distilled off at 240° C. until the remaining mixture had a hydroxyl number of 884 mg KOH/g.

A mixture of the polyglycerol thus obtained (243.5 g, 0.32 mol) and stearic acid and palmitic acid in a ratio of 1:1 (85.42 g, 60 mol) and caprylic acid and capric acid in a ratio of 60:40 (3.82 g, 0.025 mol) was heated up to 240° C. over the course of 3 h with the introduction of nitrogen and the mixture was then stirred at this temperature and the water which formed was continuously removed until an acid number of ≦1.0 was reached and the mixture was clear and homogeneous at 240° C.

### Synthesis Example 5

### Inventive

A mixture of esters obtained as described in presynthesis example 1 (18.75 g), in presynthesis example 2 (33.75 g) and in presynthesis example 8 (97.5 g) was heated to 80° C. and the mixture then stirred at this temperature for 3 h.

The polyglycerol ester thus obtained, after its complete hydrolysis, has a degree of polymerization of the polyglycerol of ca. 3.8.

### Application Examples of Inventive Compositions vs. Non-Inventive Compositions

All concentrations in the application examples are given in percent by weight. Customary homogenization processes known to the person skilled in the art were used to produce the emulsions.

The emulsions were therefore produced typically by heating oil phase and water phase to 70-75° C. Subsequently, either the oil phase was stirred into the water phase, or oil phase and water phase were combined without stirring. The mixture was then homogenized using a suitable homogenizer (e.g. Ultraturrax) for about 1-2 minutes.

Stabilizing polymers were stirred into the emulsion at temperatures of 50-60° C., either as constituent of the oil phase (e.g. guar derivatives) or as an aqueous suspension (e.g. cellulose derivatives). The mixture was then briefly homogenized.

Addition of further ingredients (e.g. preservatives, active ingredients) was preferably carried out at 40° C. If the formulations were preserved with organic acids, the pH of the emulsions was adjusted to about 5.

1) Differentiation of Performance vs. the Prior Art

These experiments show that the emulsifiers according to the invention have advantages with regard to emulsion stability. As representatives of PEG-free O/W emulsifiers, the combination of Methyl Glucose Sesquistearate/Polyglyceryl-4 Laurate and Polyglyceryl-4 Laurate/Succinate (and) Aqua was selected in this case. In addition, an emulsifier according to EP2705832 was used in the formulations 1-8, 2-8 and 3-8, and an emulsifier according to WO2015132053 was used in formulations 1-8, 2-8 and 3-8.

To test the storage stability of the emulsions, these were stored for three months at room temperature and 40° C. To assess the low-temperature stability, moreover, they were stored for one month at −5° C., and three freeze-thaw cycles of 25° C./−15° C./25° C. were carried out. Considerable changes in the appearance or the consistency, and in particular oil or water separations, were weighted as criteria for instability.

A) Aluminium salt-containing antiperspirant/deodorant formulation

| | Formulation | | | | | | | | | |
|---|---|---|---|---|---|---|---|---|---|---|
| | 1-1 | | | 1-4 | 1-5 | 1-6 | 1-7 | 1-8 | 1-9 | 1-10 |
| | Non- | 1-2 | 1-3 | Non- | Non- | Non- | Non- | Non- | Non- | Non- |
| | inven- | Inven- | Inven- | inven- | inven- | inven- | inven- | inven- | inven- | inven- |
| | tive | tive | tive | tive | tive | tive | tive | tive | tive | tive |
| Non-inventive | 3.2 | | | | | | | | | |
| emulsifier | | | | | | | | | | |
| according to | | | | | | | | | | |
| synthesis | | | | | | | | | | |
| example 1 | | | | | | | | | | |
| Inventive | | 3.2 | | | | | | | 3.2 | |
| emulsifier | | | | | | | | | | |
| according to | | | | | | | | | | |
| synthesis | | | | | | | | | | |
| example 2 | | | | | | | | | | |
| Inventive | | | 3.2 | | | | | | | |
| emulsifier | | | | | | | | | | |
| according to | | | | | | | | | | |
| synthesis | | | | | | | | | | |
| example 3 | | | | | | | | | | |
| Methyl Glucose | | | | 1.75 | 1.75 | | | | | |
| Sesquistearate 1) | | | | | | | | | | |
| Polyglyceryl-4 | | | | 0.25 | 0.25 | | | | | |
| Laurate2) | | | | | | | | | | |
| Polyglyceryl-4 | | | | | | 2.1 | 2.1 | | | |
| Laurate/Succinate | | | | | | | | | | |
| (and) Aqua 3) | | | | | | | | | | |
| Polyglyceryl-6 | | | | | | | | 3.2 | | |
| Stearate; | | | | | | | | | | |
| Polyglyceryl-6 | | | | | | | | | | |
| Behenate 29) | | | | | | | | | | |
| Polyglycerol ester | | | | | | | | | | 3.2 |
| corresponding | | | | | | | | | | |
| to synthesis | | | | | | | | | | |
| example 1 in | | | | | | | | | | |
| WO2015132053 | | | | | | | | | | |
| C18-C22 | 0.2 | 0.2 | 0.2 | 0.5 | | 0.5 | | 0.2 | | 0.2 |
| Hydroxyalkyl | | | | | | | | | | |
| Hydroxypropyl | | | | | | | | | | |
| Guar 4) | | | | | | | | | | |
| Hydroxyethyl- | | | | | 0.5 | | 0.5 | | | |
| cellulose 5) | | | | | | | | | | |
| Hydroxypropyl | | | | | | | | | 0.2 | |
| Guar 30) | | | | | | | | | | |
| Isoamyl | 5.4 | 5.4 | 5.4 | 7.0 | 7.0 | 5.4 | 5.4 | 5.4 | 5.4 | 5.4 |
| Cocoate 6) | | | | | | | | | | |
| Water | to 100 | to 100 | to 100 | to 100 | to 100 | to 100 | to 100 | to 100 | to 100 | to 100 |
| Aluminum | 20.0 | 20.0 | 20.0 | 15.0 | 15.0 | 20.0 | 20.0 | 20.0 | 20.0 | 20.0 |
| Chlorohydrate | | | | | | | | | | |
| (50% aq.) 7 | | | | | | | | | | |
| Methyliso- | 0.8 | 0.8 | 0.8 | 0.8 | 0.8 | 0.8 | 0.8 | 0.8 | 0.8 | 0.8 |
| thiazolinone, | | | | | | | | | | |
| Methylparaben, | | | | | | | | | | |
| Ethylparaben; | | | | | | | | | | |
| Dipropylene | | | | | | | | | | |
| Glycol 8) | | | | | | | | | | |
| Actual pH | 4.2 | 4.2 | 4.2 | 4.2 | 4.2 | 4.2 | 4.2 | 4.2 | 4.2 | 4.2 |
| Viscosity [Pas] | 21 | 5 | 9 | 22 | 16 | “water- | “water- | 7 | “water- | 5 |
| (Brookfield RVT, | | | | | | thin” | thin” | | thin” | |
| Spindle 4, 5 rpm) | | | | | | | | | | |
| Stability | Unstable, | Stable | Stable | Unstable, | Unstable, | Unstable, | Unstable, | Unstable, | Unstable, | Unstable, |
| | water | | | water | water | water | water | water | water | water |
| | separation | | | separation | separation | separation | separation | separation | separation | separation |
| | after 2 | | | after 2 | after 2 | after 2 | after 2 | after 1 month | after 2 | after 2 |
| | days at RT | | | weeks at RT | days at RT | days at RT | days at RT | at 40° C., | days at RT | days at RT, |
| | | | | | | | | or after 1 | | phase |
| | | | | | | | | month | | separation |
| | | | | | | | | at −5° C. | | after 2 |
| | | | | | | | | | | days at |
| | | | | | | | | | | 40° C. |

1) TEGO Care PS (Evonik Industries AG)
2)TEGO Care PL 4 (Evonik Industries AG)
29) Polyglycerol ester corresponding to synthesis example 1 in EP2705832
30) ESAFLOR HDR (Lamberti S.p.A.)
3) NatraGem E145 (Croda Int. Plc)
4) ESAFLOR HM 22 (Lamberti S.p.A.)
5) Natrosol 250 HHR (Ashland Specialty Ingredients)
6) TEGOSOFT AC (Evonik Industries AG)
7 Locron LIC (Clariant AG)
8) Microcare MEM (Thor)

B) Aluminum-free deodorant formulation without antiperspirant active ingredients

| | Formulation | | | | | | | | | |
|---|---|---|---|---|---|---|---|---|---|---|
| | 2-1 | 2-2 | 2-3 | 2-4 | 2-5 | 2-6 | 2-7 | 2-8 | 2-9 | 2-10 |
| | Non-inv. | Inv. | Inv. | Non-inv. | Non-inv. | Non-inv. | Non-inv. | Non-inv. | Non-inv. | Non-inv. |
| | mix. | mix. | mix. | mix. | mix. | mix. | mix. | mix. | mix. | mix. |
| Non-inventive | 3.2 | | | | | | | | | |
| emulsifier | | | | | | | | | | |
| according to | | | | | | | | | | |
| synthesis | | | | | | | | | | |
| example 1 | | | | | | | | | | |
| Inventive | | 3.2 | | | | | | | | |
| emulsifier | | | | | | | | | | |
| according to | | | | | | | | | | |
| synthesis | | | | | | | | | | |
| example 2 | | | | | | | | | | |
| Inventive | | | 3.2 | | | | | | 3.2 | |
| emulsifier | | | | | | | | | | |
| according to | | | | | | | | | | |
| synthesis | | | | | | | | | | |
| example 3 | | | | | | | | | | |
| Methyl Glucose | | | | 2.8 | 2.8 | | | | | |
| Sesquistearate 1) | | | | | | | | | | |
| Polyglyceryl-4 | | | | 0.4 | 0.4 | | | | | |
| Laurate2) | | | | | | | | | | |
| Polyglyceryl-4 | | | | | | 3.2 | 3.2 | | | |
| Laurate/Succinate | | | | | | | | | | |
| (and) Aqua 3) | | | | | | | | | | |
| Polyglyceryl-6 | | | | | | | | 3.2 | | |
| Stearate; | | | | | | | | | | |
| Polyglyceryl-6 | | | | | | | | | | |
| Behenate 29) | | | | | | | | | | |
| Polyglycerol ester | | | | | | | | | | 3.2 |
| corresponding | | | | | | | | | | |
| to synthesis | | | | | | | | | | |
| example 1 in | | | | | | | | | | |
| WO2015132053 | | | | | | | | | | |
| C18-C22 | 0.15 | 0.15 | 0.15 | 0.15 | | 0.15 | | 0.15 | | 0.15 |
| Hydroxyalkyl | | | | | | | | | | |
| Hydroxypropyl | | | | | | | | | | |
| Guar 4) | | | | | | | | | | |
| Hydroxyethyl- | | | | | 0.15 | | 0.15 | | | |
| cellulose 5) | | | | | | | | | | |
| Hydroxypropyl | | | | | | | | | 0.15 | |
| Guar 30) | | | | | | | | | | |
| Polyglyceryl-3 | 0.5 | 0.5 | 0.5 | 0.5 | 0.5 | 0.5 | 0.5 | 0.5 | 0.5 | 0.5 |
| Caprylate 9) | | | | | | | | | | |
| Zinc | 1.0 | 1.0 | 1.0 | 1.0 | 1.0 | 1.0 | 1.0 | 1.0 | 1.0 | 1.0 |
| Ricinoleate 10) | | | | | | | | | | |
| Caprylic/Capric | 5.65 | 5.65 | 5.65 | 5.65 | 5.65 | 5.65 | 5.65 | 5.65 | 5.65 | 5.65 |
| Triglyceride | | | | | | | | | | |
| Water | to 100 | to 100 | to 100 | to 100 | to 100 | to 100 | to 100 | to 100 | to 100 | to 100 |
| Glycerol | 3.0 | 3.0 | 3.0 | 3.0 | 3.0 | 3.0 | 3.0 | 3.0 | 3.0 | 3.0 |
| Benzyl Alcohol, | 1.0 | 1.0 | 1.0 | 1.0 | 1.0 | 1.0 | 1.0 | 1.0 | 1.0 | 1.0 |
| Benzoic Acid, | | | | | | | | | | |
| Sorbic Acid 11) | | | | | | | | | | |
| Citric acid | q.s. | q.s. | q.s. | q.s. | q.s. | q.s. | q.s. | q.s. | q.s. | q.s. |
| (50% aq.) | | | | | | | | | | |
| pH | 5.0 | 5.0 | 5.0 | 5.0 | 5.0 | 5.0 | 5.0 | 5.0 | 5.0 | 5.0 |
| Viscosity [Pas] | 7 | 4 | 5 | 18 | 10 | “water- | “water- | 2 | 2 | 5 |
| (Brookfield RVT, | | | | | | thin” | thin” | | | |
| Spindle 4, 5 rpm) | | | | | | | | | | |
| Stability | Unstable, | Stable | Stable | Excessive | Unstable, | Unstable, | Unstable, | Unstable, | Unstable, | Unstable, |
| | water | | | viscosity, | water | phase | phase | water | water | phase |
| | separation | | | not stable | separation | separation | separation | separation | separation | separation |
| | after 3 | | | to freezing | on warm | after | after | after 3 | after 1 | after 1 |
| | months at | | | (−5° C. | storage | one day | one day | months | month at | month at |
| | 40° C. | | | and −15° C.) | (40° C.) | | | at RT | 40° C. | 40° C. |

9) TEGO Cosmo P 813 (Evonik Industries AG)
10) TEGODEO PY 88 G (Evonik Industries AG)
11) Rokonsal BSB-N (Ashland Specialty Ingredients)

C) O/W Deodorant emulsion comprising potassium alum

| | Formulation | | | | | | | | | |
|---|---|---|---|---|---|---|---|---|---|---|
| | 3-1 | 3-2 | 3-3 | 3-4 | 3-5 | 3-6 | 3-7 | 3-8 | 3-9 | 3-10 |
| | Non-inv. | Inv. | Inv. | Non-inv. | Non-inv. | Non-inv. | Non-inv. | Non-inv. | Non-inv. | Non-inv. |
| | mix. | mix. | mix. | mix. | mix. | mix. | mix. | mix. | mix. | mix. |
| Non-inventive | 4.8 | | | | | | | | | |
| emulsifier | | | | | | | | | | |
| according to | | | | | | | | | | |
| synthesis | | | | | | | | | | |
| example 1 | | | | | | | | | | |
| Inventive | | 4.8 | | | | | | | 4.8 | |
| emulsifier | | | | | | | | | | |
| according to | | | | | | | | | | |
| synthesis | | | | | | | | | | |
| example 2 | | | | | | | | | | |
| Inventive | | | 4.8 | | | | | | | |
| emulsifier | | | | | | | | | | |
| according to | | | | | | | | | | |
| synthesis | | | | | | | | | | |
| example 3 | | | | | | | | | | |
| Methyl Glucose | | | | 4.2 | 4.2 | | | | | |
| Sesquistearate 1) | | | | | | | | | | |
| Polyglyceryl-4 | | | | 0.6 | 0.6 | | | | | |
| Laurate2) | | | | | | | | | | |
| Polyglyceryl-4 | | | | | | 4.8 | 4.8 | | | |
| Laurate/Succinate | | | | | | | | | | |
| (and) Aqua 3) | | | | | | | | | | |
| Polyglyceryl-6 | | | | | | | | 4.8 | | |
| Stearate; | | | | | | | | | | |
| Polyglyceryl-6 | | | | | | | | | | |
| Behenate 29) | | | | | | | | | | |
| Polyglycerol ester | | | | | | | | | | 4.8 |
| corresponding | | | | | | | | | | |
| to synthesis | | | | | | | | | | |
| example 1 in | | | | | | | | | | |
| WO2015132053 | | | | | | | | | | |
| C18-C22 | 0.25 | 0.25 | 0.25 | 0.25 | | 0.25 | | 0.25 | | 0.25 |
| Hydroxyalkyl | | | | | | | | | | |
| Hydroxypropyl | | | | | | | | | | |
| Guar 4) | | | | | | | | | | |
| Hydroxyethyl- | | | | | 0.25 | | 0.25 | | | |
| cellulose 5) | | | | | | | | | | |
| Hydroxypropyl | | | | | | | | | 0.25 | |
| Guar 30) | | | | | | | | | | |
| Isopropyl | 5.0 | 5.0 | 5.0 | 5.0 | 5.0 | 5.0 | 5.0 | 5.0 | 5.0 | 5.0 |
| Palmitate | | | | | | | | | | |
| Water | to 100 | to 100 | to 100 | to 100 | to 100 | to 100 | to 100 | to 100 | to 100 | to 100 |
| Glycerol | 3.0 | 3.0 | 3.0 | 3.0 | 3.0 | 3.0 | 3.0 | 3.0 | 3.0 | 3.0 |
| Potassium | 5.0 | 5.0 | 5.0 | 5.0 | 5.0 | 5.0 | 5.0 | 5.0 | 5.0 | 5.0 |
| alum | | | | | | | | | | |
| Methyliso- | 0.8 | 0.8 | 0.8 | 0.8 | 0.8 | 0.8 | 0.8 | 0.8 | 0.8 | 0.8 |
| thiazolinone, | | | | | | | | | | |
| Methylparaben, | | | | | | | | | | |
| Ethylparaben; | | | | | | | | | | |
| Dipropylene | | | | | | | | | | |
| Glycol 8) | | | | | | | | | | |
| Actual pH | 3.4 | 3.4 | 3.4 | 3.4 | 3.4 | 3.4 | 3.4 | 3.4 | 3.4 | 3.4 |
| Viscosity [Pas] | 11 → 23 | 3 | 3 | 4 | — | — | — | 11 → 34 | — | 5 |
| (Brookfield RVT, | | | | | | | | | | |
| Spindle 4, 5 rpm) | | | | | | | | | | |
| Stability | Excessively | Stable | Stable | Unstable, | Unstable, | Unstable, | Unstable, | excessively | Unstable, | Unstable, |
| | high | | | water | phase | phase | phase | high | phase | water |
| | viscosity | | | separation | separation | separation | separation | viscosity | separation | separation |
| | increase | | | after 1 | 3 h after | 3 h after | 3 h after | increase | 3 h after | after 2 |
| | on storage | | | month at | preparation | preparation | preparation | on storage | preparation | days at |
| | | | | 40° C. | | | | | | 40° C. |

In all three comparative formulations from the application fields of antiperspirant/deodorant or roll-on deodorant comprising various active ingredients, the emulsifiers according to the invention allow the formulation of a stable emulsion, whereas the non-inventive emulsifiers and the representatives of the prior art do not enable any stable emulsions. The use of non-inventively modified guar and emulsifiers according to EP2705832 or WO2015132053 likewise leads to emulsions that are unstable on storage.

Formulations 1-2 and 1-9 were investigated with regard to their ability to form a yield point. The measurements were carried out using a rheometer from Anton Paar, model MCR 301, plate—plate (40 mm) geometry with a 1 mm gap at a temperature of 25° C. and 1 pressure bar. Storage modulus and loss modulus were determined for the emulsions prepared according to inventive formulation 1-2 or non-inventive formulation 1-9. The samples were analysed at a constant load of 0.02 Pa over the frequency range of 0.005 to 90 Hz. The rheological yield point is defined in that, in the measured frequency range, the storage modulus (G′) always has higher values than than the loss modulus (G″) (see FIG. 1).

Further application examples beyond the antiperspirant/deodorant field.

These examples show that the compositions according to the invention can be used in a large number of cosmetic formulations.

Sun Care Spray SPF 30

| Formulation | 4 |
|---|---|
| Inventive emulsifier according to synthesis | 4.0 |
| example 4 | |
| Phenoxyethyl Caprylate¹²⁾ | 3.2 |
| Isopropyl Palmitate | 2.0 |
| Bis-Ethylhexyloxyphenol Methoxyphenyl | 3.0 |
| Triazine¹³⁾ | |
| Butyl Methoxydibenzoylmethane | 2.0 |
| EHC | 2.0 |
| Ethylhexyl Salicylate | 4.0 |
| Octocrylene | 4.0 |
| Glycerol | 3.0 |
| Water | to 100 |
| Carbomer suspension 1¹⁴⁾ | 1.0 |
| Tris(hydroxymethyl)aminomethane (30% | 0.6 |
| aq.) | |
| UV filter solution¹⁵⁾ | 10.0 |
| Methylisothiazolinone, Methylparaben, | 0.8 |
| Ethylparaben; Dipropylene Glycol⁸⁾ | |

¹²⁾TEGOSOFT XC (Evonik Industries AG)
¹³⁾Tinosorb S (BASF SE)
¹⁴⁾Acrylates/C10-30 Alkyl Acrylate Crosspolymer (TEGO Carbomer 341ER, Evonik Industries AG) 20% in Phenoxyethyl Caprylate
¹⁵⁾20% Phenylbenzimidazole Sulfonic Acid, 8.8% Tris(hydroxymethyl)aminomethane, demineralized water to 100%

Body lotion

| Formulation | 5 |
|---|---|
| Inventive emulsifier | 4.0 |
| according to synthesis | |
| example 5 | |
| Isoamyl Cocoate⁶⁾ | 2.5 |
| Caprylic/Capric | 3.5 |
| Triglyceride | |
| Water | to 100 |
| Creatine¹⁶⁾ | 0.5 |
| Carbomer suspension 2¹⁷⁾ | 1.0 |
| Sodium hydroxide (10% | 0.6 |
| aq.) | |
| Phenoxyethanol, | 0.7 |
| Ethylhexylglycerol¹⁸⁾ | |

¹⁶⁾TEGO Cosmo C 100 (Evonik Industries AG)
¹⁷⁾Carbomer (TEGO Carbomer 141, Evonik Industries AG) 20% in Ethylhexyl Stearate
¹⁸⁾Euxyl PE 9010 (Schulke & Mayr GmbH)

Natural care cream

| Formulation | 6 |
|---|---|
| Inventive emulsifier according to | 6.0 |
| synthesis example 2 | |
| Caprylic/Capric Triglyceride | 8.0 |
| Isopropyl Palmitate | 11.0 |
| Prunus Amygdalus Dulcis (Sweet | 10.0 |
| Almond) Oil | |
| Water | to 100 |
| Glycerol | 3.0 |
| Sodium hydroxide (10% aq.) | 0.2 |
| Benzyl Alcohol, Benzoic Acid, Sorbic | 0.80 |
| Acid¹¹⁾ | |

Anti-Aging Cream

| Formulation | 7 |
|---|---|
| Inventive emulsifier according to | 6.0 |
| synthesis example 4 | |
| Caprylic/Capric Triglyceride | 9.5 |
| C12-15 Alkyl Benzoate | 9.5 |
| Water | to 100 |
| Glycerol | 3.0 |
| Tetrapeptide-21; Glycerol; Butylene | 4.0 |
| Glycol; Aqua¹⁹⁾ | |
| Sodium Hyaluronate²⁰⁾ | 0.1 |
| Hydrolyzed Hyaluronic Acid²¹⁾ | 0.1 |
| Aqua; Ethylhexyl Stearate; Sodium | 5.0 |
| Hyaluronate Crosspolymer; | |
| Polyglyceryl-4 | |
| Diisostearate/Polyhydroxystearate/Sebacate; | |
| Sodium Isostearate²²⁾ | |
| Methylisothiazolinone, Methylparaben, | 0.8 |
| Ethylparaben; Dipropylene Glycol⁸⁾ | |

¹⁹⁾TEGO PEP 4-17 (Evonik Industries)
²⁰⁾HyaCare (Evonik Industries)
²¹⁾HyaCare 50 (Evonik Industries)
²²⁾HyaCare Filler CL (Evonik Industries)

O/W Foundation

| Formulation | 8 |
|---|---|
| Inventive emulsifier according to | 6.0 |
| synthesis example 3 | |
| Myristyl Myristate | 2.0 |
| Isopropyl Myristate | 6.0 |
| Decyl Cocoate | 6.0 |
| Cetyl Ricinoleate | 1.0 |
| Water | to 100 |
| Glycerol | 1.0 |
| Titanium Dioxide²³⁾ | 8.0 |
| Iron Oxides²⁴⁾ | 0.9 |
| Iron Oxides²⁵⁾ | 0.2 |
| Iron Oxides²⁶⁾ | 0.4 |
| Iron Oxides²⁷⁾ | 0.1 |
| Cellulose²⁸⁾ | 2.0 |
| Sodium hydroxide (10% aq.) | 0.2 |
| Benzyl Alcohol, Benzoic Acid, Sorbic | 1.0 |
| Acid¹¹⁾ | |

²³⁾Hombitan AC 360 (Sachtleben)
²⁴⁾Sicovit Yellow 10 E 172 (Rockwood Pigments)
²⁵⁾Sicovit Red 30 E 172 (Rockwood Pigments)
²⁶⁾Sicovit Brown 70 E 172 (Rockwood Pigments)
²⁷⁾Sicovit Black 80 E 172 (Rockwood Pigments)
²⁸⁾TEGO Feel Green (Evonik Industries AG)

1. A composition comprising
A) polyglycerol ester,
which, after its complete hydrolysis, releases
a) at least one carboxylic acid having 6 to 14 carbon atoms,
b) at least one carboxylic acid having 16 to 20 carbon atoms,
c) at least one carboxylic acid having 22 to 28 carbon atoms,
and
which, after its complete hydrolysis, releases a polyglycerol having an average degree of polymerization of from 3.0 to 5.0,
and
B) at least one hydroxyalkyl-modified guar,
wherein the hydroxyalkyl-modified guar has been modified with hydroxyalkyl groups having 12 to 26 carbon atoms.

2. The composition according to claim 1, wherein the polyglycerol ester, after its complete hydrolysis, releases an average (number average) per mole of polyglycerol ester of
from 0.01 to 0.07 mol of at least one carboxylic acid a)
from 0.10 to 1.70 mol of at least one carboxylic acid b)
from 0.01 to 0.80 mol of at least one carboxylic acid c).

3. The composition according to claim 1, wherein the molar ratio of carboxylic acid a) to carboxylic acid b) to carboxylic acid c) obtained after complete hydrolysis of the polyglycerol ester is
6 to 1.4:16.5 to 20.5:3.0 to 4.8.

4. The composition according to claim 1, wherein the carboxylic acids a), b) and c) are selected from linear, saturated, unsubstituted carboxylic acids.

5. The composition according to claim 1, wherein carboxylic acid a) is selected from caprylic acid and capric acid, carboxylic acid b) is selected from stearic acid and palmitic acid and carboxylic acid c) is behenic acid.

6. The composition according to claim 1 wherein the polyglycerol released after complete hydrolysis of the polyglycerol ester has a mass ratio of glycerol to diglycerol of greater than 1.

7. The composition according to claim 1, wherein the hydroxyalkyl-modified guar is modified with hydroxyalkyl groups having 18 to 24 carbon atoms.

8. The composition according to claim 1, wherein the hydroxyalkyl-modified guar has additionally been hydroxypropyl-modified.

9. The composition according to claim 1, wherein said composition additionally comprises
C) at least one active ingredient selected from deodorant active ingredient and antiperspirant active ingredient.

10. The composition according to claim 1, wherein the antiperspirant active ingredient is selected from the group comprising consisting of, aluminium salts and zirconium salts, and the deodorant active ingredient from the group of zinc salts, in particular aluminium chlorohydrate and zinc ricinoleate.

11. The composition according to claim 1, wherein said composition is an emulsion, particularly an oil-in-water emulsion.

12. The composition according to claim 11, wherein said composition has a viscosity in a range from 500 to 20 000 mPas.

13. The composition according to claim 1, wherein said composition formulation is essentially free from polyglycol ethers and free from alkoxylated compounds.

14. The composition of claim 1 comprising
A) polyglycerol ester,
which, after its complete hydrolysis, releases
a) at least one carboxylic acid having 8 to 12 carbon atoms,
b) at least one carboxylic acid having 18 to 20 carbon atoms,
c) at least one carboxylic acid having 22 to 24 carbon atoms.

15. The composition of claim 1 wherein after its complete hydrolysis, releases a polyglycerol having an average degree of polymerization of from 3.3 to 4.7.

16. The composition according to claim 1, wherein the polyglycerol ester, after its complete hydrolysis, releases an average (number average) per mole of polyglycerol ester of
from 0.01 to 0.50 mol of at least one carboxylic acid a)
from 0.30 to 1.50 mol of at least one carboxylic acid b)
from 0.01 to 0.60 mol of at least one carboxylic acid c).

17. The composition according to claim 1, wherein the molar ratio of carboxylic acid a) to carboxylic acid b) to carboxylic acid c) obtained after complete hydrolysis of the polyglycerol ester is 0.8 to 1.2:17.5 to 19.5:3.5 to 4.3.

18. The composition according to claim 11, wherein said composition has a viscosity in a range from 1500 to 10 000 mPas.

19. The composition according to claim 1, wherein the hydroxyalkyl-modified guar is modified with hydroxyalkyl groups having 20 to 22 carbon atoms.

20. The composition according to claim 1 wherein the polyglycerol released after complete hydrolysis of the polyglycerol ester has a mass ratio of glycerol to diglycerol of greater than 1.2.
